(19) United States
(12) Patent Application Publication
(10) Pub. No.: US 2003/0100828 A1
(43) Pub. Date: May 29, 2003

(54) Method for minimally invasive prostate tumor treatment

(75) Inventors: Karl Engelhard, Erlangen (DE); Rainer Kuth, Herzogenaurach (DE); Hans-Peter Hollenbach, Eggolsheim (DE)

Correspondence Address: SCHIFF HARDIN & WAITE, 6600 SEARS TOWER, 233 S WACKER DR, CHICAGO, IL, 60606-6473, US

(73) Assignee: Siemens Aktiengesellschaft

(21) Appl. No.: 09/997,346

(22) Filed: Nov. 29, 2001

(51) Int. Cl. A61B 5/05

(52) U.S. Cl. 600/411

(57) ABSTRACT

In a method for minimally invasive treatment of prostate cancer, a patient with a prostate pathology is subjected to a magnetic resonance-guided biopsy procedure, and a rapid histology is performed on the biopsy sample. If the histology result is positive, the patient is then subjected to a magnetic resonance-guided injection of a therapeutic agent. Since the therapeutic agent is administered during observation of the patient during magnetic resonance, it can be ascertained if and when the administered therapeutic agent fully spreads to cover the pathological region or volume. After an appropriate waiting time the patient is examined in a follow-up examination and appropriate further treatment, if any, is determined based on this follow-up examination.

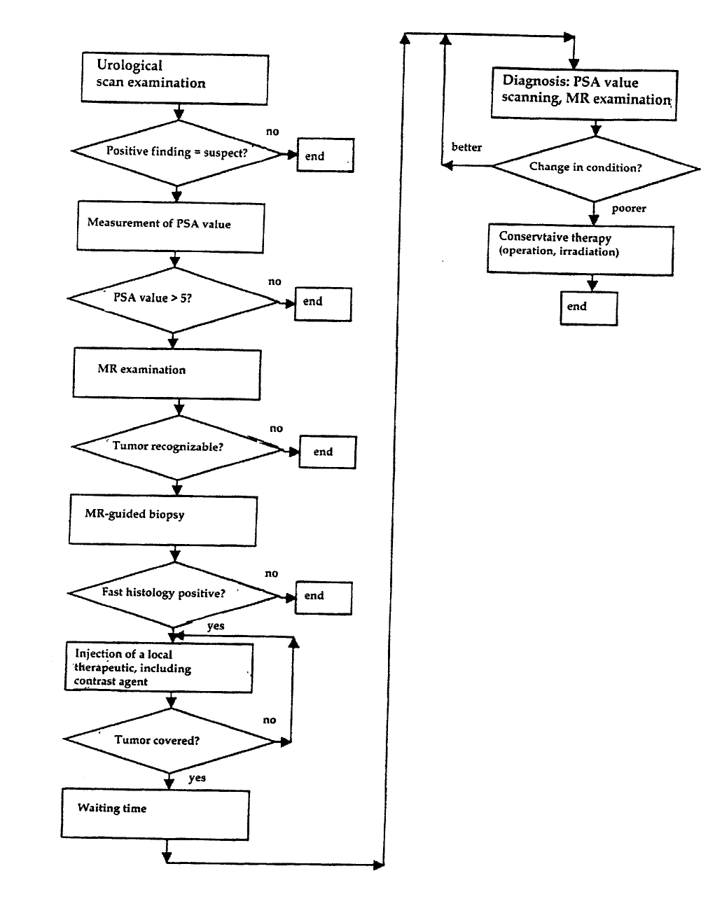

### BACKGROUND OF THE INVENTION

1. Field of the Invention

The present invention is directed to a method for minimally invasive treatment of prostate tumors, and in particular to such a method employing a magnetic resonance apparatus.

2. Description of the Prior Art

Prostate tumors represent a serious condition that is dramatically increasing in developed countries. This is for several reasons. First, an increasing number of males are reaching an age at which prostate tumors become life-threatening. Moreover, an increasing number of younger men are being diagnosed with prostate tumors.

Untreated prostate carcinoma metastasize into the bones, and can lead to a long and serious illness that is generally fatal. Conventional therapy procedures includes chemotherapy, radiation therapy and surgery. All of these types of known treatments subject the patient to significant stress, and have undesirable side effects associated therewith. Moreover, many patients are not considered suitable to receive conventional therapies of this type due to pre-existing medical conditions, such as diabetes, or due to another, unrelated therapeutic regimen, such as the patient taking blood-thinning medication, or due to physiological reasons.

A number of minimally invasive therapies are known. One such minimally invasive therapy is photodynamic therapy, in which the patient is administered a therapeutic agent that becomes toxic under the influence of light. After the therapeutic agent has been administered to the patient, the prostate tumor is observed by magnetic resonance imaging so that one or more catheters can be introduced into the prostate tumor, via which light is applied in the cancerous area with optical fibers. This activates the therapeutic agent to become toxic in the localized region of the tumor, so that the tumor is poisoned and the region or volume within which the therapeutic agent has been made toxic is limited to the region exposed to light.

### SUMMARY OF THE INVENTION

It is an object of the present invention to provide a method for treatment of a prostate tumor that minimally invasive, can be economically conducted, and subjects the patient to relatively low stress. A further object of the present invention is to provide such a method which does limit the possibility of subsequently employing more invasive therapies, if needed.

The above objects are achieved in accordance with the present invention in a method for minimally invasive prostate tumor treatment wherein a biopsy is performed on a patient with monitoring by magnetic resonance imaging, the biopsy specimen is subjected to a fast histology, if the histology result is positive, a locally acting cell toxin is immediately injected into the tumor, also with monitoring by magnetic resonance imaging to be sure that the toxin covers (spreads to) a region commensurate with the tumor. For this purpose, if the cell toxin itself does not appear clearly in a magnetic resonance image, a magnetic resonance imaging contrast agent, such Gd-DPPA in water can be mixed with the cell toxin. At appropriate intervals determined by a physician, the patient is examined and tested by one or more obtaining a PSA count, conducting an ultra-scan, or conducting a further magnetic resonance scan. If the result of this follow-up testing and examination shows an improvement in the condition of the prostate, further follow-up testing and examination can be conducted. If no change in condition is seen in the follow-up testing and examination, the aforementioned localized administration of a cell toxin under magnetic resonance observation can be repeated, or other therapeutic methods can be considered.

### DESCRIPTION OF THE DRAWINGS

The drawing is a flowchart of an embodiment of a method for minimally invasive prostate tumor treatment in accordance with the invention.

### DESCRIPTION OF THE PREFERRED EMBODIMENTS

As part of the inventive method, a patient can be subjected to a pre-screening examination to determine whether the patient is at risk of having a prostate tumor. Such pre-screening can begin with a conventional urological examination, possibly including an ultrasound scan. If the result of the this initial examination is positive, or at least constitute a basis for a suspicion that a tumor may be present, the patient's PSA value can be measured. The patient's physician can make a decision based on the physician's experience and the patient's medical history as to what PSA value will trigger the remaining steps of the method, but typically a PSA value greater than 5 will be an indication that the remaining steps should be implemented.

A patient for whom the remaining method steps are determined to be appropriate is then subjected to a magnetic resonance examination wherein, as a threshold observation, it is determined whether a tumor is recognizable in the magnetic resonance images. If so, a magnetic resonance guided biopsy is immediately conducted. Preferably while the patient remains in the magnetic resonance scanner, a rapid histology is conducted. If the histology result is negative, the patient is removed from the scanner and, as needed, follow-up examinations may be prescribed.

If the histology result is positive, the patient in the MR scanner is then injected, again under magnetic resonance guidance, with a localized therapeutic agent. By observing the spread of the therapeutic agent in the magnetic resonance images, it is determined if and when the therapeutic agent covers the tumor, i.e., has spread to a region of volume commensurate with the region or volume of the tumor.

If the therapeutic agent itself does not possess attributes so as to be clearly visible in the magnetic resonance images, the local therapeutic can be mixed with a contrast agent, such as Gd-DPPA in water.

The local therapeutic itself can be an embolizer, such as ethanol, ethoxy scleral. These local therapeutics can be administered respectively in pure form, or mixed together, or individually in mixtures with water, or together in a mixture with water.

Another example of a suitable local therapeutic is cytostatic, for example, mitomycin C, cisplatin, 5-FU fluoruracil. Again, these cytostatic therapeutic agents can be administered in pure form, or in any combination of mixtures.

A further possibility is to mix the local therapeutic with hormones.

For patient comfort, it may also be desirable to mix the local therapeutic with a local anaesthetic.

Following the magnetic resonance guided injection of the therapeutic agent, an appropriate waiting time, determined by the physician, ensues. After this waiting time, in a follow-up examination, the progress of the treatment is determined by one or more of PSA value measurement, ultrasound scanning and magnetic resonance examination. If the result of this follow-up examination and testing shows improvement, i.e., a reduction in the PSA value and/or a visibly discernable reduction in the size of the tumor, further follow-up examination can be scheduled. If no improvement is seen, the physician may prescribe a repetition of the method steps involving magnetic resonance-guide therapeutic agent injection, or may determine that a conventional therapy technique, such as chemotherapy, radiation therapy or surgery is recommnended. The inventive method does not complicate or compromise any of these conventional therapies, if it is subsequently determined that they are necessary.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for minimally invasive treatment of a prostate tumor comprising the steps of:
introducing a patient having a prostate pathology into a magnetic resonance imaging apparatus and obtaining an image of the prostate by magnetic resonance imaging;
if said prostate pathology is identifiable in said prostate image, retaining said patient in said magnetic resonance imaging apparatus and conducting a magnetic resonance-guided biopsy to obtain a biopsy sample from a region of the prostate in which said prostate pathology is identified;
immediately performing a histology on said biopsy sample to obtain a histology result;
if said histology result is positive, injecting, while said patient is in said magnetic resonance imaging apparatus, a local prostate cancer therapeutic agent into said region of said prostate with magnetic resonance guidance and obtaining at least one further magnetic resonance image to determine whether said therapeutic agent has covered a region in said prostate containing said pathology;
removing said patient from said magnetic resonance imaging apparatus and, after a waiting time, conducting a follow-up examination of said patient to determine whether a change in said prostate pathology has occurred; and
prescribing further treatment for said patient dependent on said follow-up examination.

2. A method as claimed in claim 1 comprising the additional step, before introducing said patient into said magnetic resonance imaging apparatus of conducting a pre-screening of said patient to determine whether said patient is at risk of having prostate cancer.

3. A method as claimed in claim 2 wherein said pre-screening includes measurement of a PSA value of said patient.

4. A method as claimed in claim 3 comprising the step of introducing said patient into said magnetic resonance imaging apparatus only if said patient has a PSA value greater than 5.

5. A method as claimed in claim 1 comprising maintaining said patient in said magnetic resonance imaging apparatus while said histology is being performed.

6. A method as claimed in claim 1 comprising injecting a locally acting cell toxin into said patient as said local prostate cancer therapeutic agent.

7. A method as claimed in claim 6 wherein said locally acting cell toxin is an embolizer.

8. A method as claimed in claim 7 wherein said embolizer is selected from the group consisting of pure ethanol, pure ethoxy scleral, ethanol mixed with water, ethoxy scleral mixed with water, ethanol and ethoxy scleral mixed together, and ethanol, ethoxy scleral and water mixed together.

9. A method as claimed in claim 6 wherein said locally acting cell toxin is a cytostatic therapeutic agent.

10. A method as claimed in claim 9 wherein said cytostatic therapeutic agent is selected from the group consisting of pure mitomycin C, cisplatin, 5-FU fluoruracil, and mixtures of mitomycin C, cisplatin and 5-FU fluoruracil.

11. A method as claimed in claim 1 comprising mixing said local prostate cancer therapeutic with a magnetic resonance contrast agent prior to injection into said patient.

12. A method as claimed in claim 11 comprising mixing said local prostate cancer therapeutic agent with Gd-DPPA in water as said contrast agent.

13. A method as claimed in claim 1 comprising mixing said local prostate cancer therapeutic agent with a local anaesthetic prior to injection into said patient.

14. A method as claimed in claim 1 comprising mixing said local prostate cancer therapeutic agent with hormones prior to injection into said patient.

15. A method as claimed in claim 1 comprising, in said follow-up examination, subjecting said patient to at least one of a PSA value measurement, an ultrasound scan and a magnetic resonance examination.

16. A method as claimed in claim 1 wherein the step of prescribing further treatment comprises prescribing a treatment selected from the group consisting of chemotherapy, radiation therapy and surgery.

17. A method as claimed in claim 1 wherein the step of prescribing further treatment comprises repeating the step of injecting said local prostate cancer therapeutic agent into said patient.
